(19) United States
(12) Patent Application Publication
(10) Pub. No.: US 2019/0029934 A1
(43) Pub. Date: Jan. 31, 2019

(54) SELF-HEATING AGENT FOR THE REDUCTIVE DECOLORATION OF DYED KERATINOUS FIBERS

(71) Applicant: Henkel AG & Co. KGaA, Duesseldorf (DE)

(72) Inventors: Juergen Schoepgens, Schwalmtal (DE); Torsten Lechner, Duesseldorf (DE)

(73) Assignee: Henkel AG & Co. KGaA, Duesseldorf (DE)

(21) Appl. No.: 15/922,547

(22) Filed: Mar. 15, 2018

(30) Foreign Application Priority Data
Mar. 15, 2017 (DE) 102017204289.3

(51) Int. Cl. A61K 8/23; A61K 8/22; A61K 8/25; A61Q 5/08

(52) U.S. Cl. CPC A61K 8/23; A61K 8/22; A61K 2800/592; A61Q 5/08; A61K 2800/88; A61K 8/25

(57) ABSTRACT

Kit-of-parts for the reductive decoloration of dyed keratinous fibers comprising, packaged separately from one another
(I) a first container (A) containing a cosmetic agent (a) and
(II) a second container (B) containing a cosmetic agent (b),
wherein
   the agent (a) in the first container (A)
(a1) contains one or more reducing agents from the group of formamidine sulphinic acids, sodium dithionite, zinc dithionite, potassium dithionite, hydroxymethane sulphinic acid, aminomethan sulphinic acid, cysteine, thiolactic acid, sulfanyl acetic acid, and/or ascorbic acid, and
   the agent (b) in container (B)
(b1) contains one or more oxidants from the group of hydrogen peroxide, potassium sulphate, sodium persulphate and/or ammonium persulphate, and
wherein the weight ratio of the total quantity of all reducing agents (a1) to the total quantity of all oxidants (b1)—relative to the total weight of agents (a) plus (b)—is a value of 50.0 to 4.0.

This application claims priority to German Patent Application No. DE102017204289.3, filed Mar. 15, 2017, which is incorporated herein by reference in its entirety.

### TECHNICAL FIELD

The present disclosure relates to the cosmetic sector. The present disclosure relates to a kit-of-parts (kit) for the reductive decoloration of dyed keratinous fibers comprising, packed separately from one another, the two containers (A) and (B).

Preparations for tinting and coloring hair are an important type of cosmetic agent. They can serve to tint the natural hair color to a lesser or greater degree depending on the preferences of each and every person, achieve a completely different hair color or cover unwanted color shades, such as shades of gray, for example. Routine hair dyes are formulated either on the basis of oxidation dyes or on the basis of partially-oxidizing dyes, depending on the preferred color and/or permanency of the dye. Often, combinations of oxidizing dyes and partially-oxidizing dyes are used to achieve special tints.

Dyes formulated on the basis of oxidation dyes lead to brilliant and permanent color shades. However, they do require the use of strong oxidants, such as hydrogen peroxide solutions, for example. Said dyes contain oxidative dye precursors, so-called developer components and coupler components. The developer components join together or couple with one or more coupler components to form, under the influence of oxidants or atmospheric oxygen, the actual colorants per se.

Dyes formulated on the basis of partially-oxidizing dyes are often used for achieving temporary colors. Partially-oxidizing dyes are dye molecules that coat the hair itself and do not require an oxidative process to create the color. Important representatives of this dye class include triphenylmethane dyes, azo dyes, anthraquinone dyes or nitrobenzene dyes, each of which can carry cationic or anionic groups.

With all said dyeing methods, however, the color may need to be reversed, either whole or in part, for various reasons. A partial removal of the color may be the ideal solution, for example, if the color result has a darker effect on the fibers than desired. On the other hand, a complete removal of the color may be desired in some cases. It is conceivable, for example, that the hair is to be colored or tinted in a particular way for a specific occasion, and the original color is to be restored after a few days.

Technical literature also discloses decolorizing agents and methods. The oxidative decoloration of dyed hair, by employing a routine blonding agent for example, is a well-known method from the prior art. With this process, however, the fibers can be damaged through the use of strong oxidants.

Moreover, reductive processes for decoloration have already been described. European Patent Application EP 1300136 A2 discloses, for example, a method for hair treatment, wherein the hair is colored in a first step and then reductively decolored again in a second step. Said reductive decoloration is achieved by employing a formulation containing a diothine salt and a tenside. In WO 2008/055756 A2, the reductive decoloration of keratinous fibers is achieved using a mixture formed from a reducing agent and an absorption agent.

When reductive decolorizing agents are used, the decoloration effect is achieved by reducing the dyes located on the keratinous fibers and/or hair. The reduction process usually involves converting the dyes to their reduced leuco forms. This method involves reducing the double bonds present in the dyes, thus interrupting the chromophoric system of the dyes and converting the dye into a colorless form.

The reduction of dyes usually requires strong reducing agents. These reducing agents are highly-reactive bonds, which are often unstable in hydrous solutions and—decompose at different rates depending on the pH value of the solution. For example, the reductive decolorizing agent sodium dithionite known from the prior art is sensitive to atmospheric oxygen and decomposes slowly in hydrous solutions. These decomposition reactions can be decelerated by increasing the pH value. Setting to a weak alkali pH value stabilizes hydrous dithionite solutions, and therefore the solution can be stored for several weeks or even months by eliminating oxygen. If, however, reductive decolorizing agents are to be stored for longer periods, or the high storage temperatures prevail, packaging in a solution, more particularly in a hydrous solution, is not the method of choice.

Other reducing agents, such as formamidine sulphinic acid for example, are unstable in hydrous solutions, and so suitable methods for preparing the reducing agents in a storable form are still being sought. In WO 2016/005114 A1, the reducing agent is worked into a fatty paste, for example. Said paste is then mixed with a hydrous formation shortly before use to produce the ready-to-use decolorizing agent. In the anhydrous environment, the reducing agent is now stable when stored for a much longer period.

Although the aforementioned packaging method is considered good progress with respect to the stability of the formulation, some requirements for the application process remain unfulfilled. Above all, the inadequate solubility of the reducing agent in the hydrous formulation proves problematic in this context. For example, formamidine sulphinic acid, also referred to as thiourea dioxide, has an especially poor water solubility of 27 g/l (measured at 20° C.).

If the user now mixes the reducing agent component (paste) with the hydrous component, he must—in order to obtain a homogeneous application mixture—mix the two components together either for an extremely long time, rigorously stir or shake the mixture or heat up the application mixture. All these options are inconvenient for the user and pose the risk that only incomplete mixing is achieved, despite his efforts. If this inhomogeneous application mixture is applied to the hair, it has in turn a disadvantageous effect on the uniformity of the decoloration result.

### BRIEF SUMMARY

Kits-of-parts for the reductive decoloration of dyed keratinous fibers, ready-to-use agents for the reductive decoloration of dyed fibers, and methods for the reductive decoloration of dyed keratinous fibers are provided herein. In an embodiment, a kit-of-parts for the reductive decoloration of dyed keratinous fibers includes, packaged separately from one another, (I) a first container (A) including a cosmetic agent (a) and (II) a second container (B) including a cosmetic agent (b). The agent (a) in the first container (A) includes (a1) one or more reducing agents chosen from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid) (thioglycol acid) and/or ascorbic acid. The agent (b) in container (B) includes (b1) one or more oxidants chosen from the group of hydrogen peroxide, potassium sulphate, sodium persulphate and/or ammonium persulphate. The weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

In another embodiment, a ready-to-use agent for the reductive decoloration of dyed keratinous fibers includes (a1) one or more reducing agents chosen from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid) (thioglycol acid) and/or ascorbic acid, and (b1) one or more oxidants chosen from the group of hydrogen peroxide, potassium sulphate, sodium persulphate and/or ammonium persulphate. The weight ratio of the total quantity of all reducing agents (a1) contained in the agent to the total quantity of all oxidants (b1) contained in the agent—relative to the total weight of agent—is a value of from about 50.0 to about 4.0.

In another embodiment, a method for the reductive decoloration of dyed keratinous fibers includes the following steps in the stated sequence. A ready-to-use decolorizing agent is produced by mixing a first agent (a) with a second agent (b). Agent (a) includes (a1) one or more reducing agents chosen from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid, and/or ascorbic acid. Agent (b) includes (b1) one or more oxidants chosen from the group of hydrogen peroxide, potassium sulphate, sodium persulphate and/or ammonium persulphate. The ready-to-use decolorizing agent is applied on dyed keratinous fibers. The decolorizing agent is then allowed to take effect. The decolorizing agent is then rinsed out of the keratinous fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure relates to a kit-of-parts (kit) for the reductive decoloration of dyed keratinous fibers comprising, packed separately from one another, the two containers (A) and (B). Container (A) contains a first cosmetic agent (a) having one or more reducing agents. Said reducing agents are suitable for the reductive decoloration of artificially-dyed hair. Said first agent (a) is preferably anhydrous. Container (B) contains a second cosmetic agent (b), which preferably constitutes a hydrous formulation. Said second agent (b) contains one of more oxidants. An essential feature of the present disclosure is that the reducing agent and the oxidants are contained in the two separate containers of the kit in a highly-specific ratio. The reducing agent to oxidant ratio is from about 4 to about 50 to 1.

A further subject matter of the present disclosure is a ready-to-use agent for the reductive decoloration of dyed keratinous fibers, which is obtained by mixing the two agents (a) and (b) described above.

A third subject matter of the present disclosure is a method for the reductive decoloration of dyed keratinous fibers, wherein the kit-of parts and/or the ready-to-use decoloration agent described above are used.

The present disclosure therefore addresses the problem of preparing new packaging possibilities, agents and methods, which allow convenient, uniform ad fast production of a ready-to-use decolorizing agent. The decolorizing agent thus produced is supposed to decolor the keratinous fibers in the most uniform and effective way possible. Moreover, the decolorizing agent is supposed to be characterized by a high storage stability, and also have a high decoloration effect even after being stored for long periods at high temperatures.

It has now surprisingly emerged that the aforementioned problem can be excellently solved by packaging the reductive decolorizing agent in the form of a kit-of-parts. This kit-of-parts comprises two containers (A) and (B), packaged separately from one another, and each containing the cosmetic agents (a) and (b). The agent (a) contains at least one reducing agent and is preferably anhydrous. Agent (b) is a hydrous cosmetic carrier, which contains small quantities of oxidants.

To produce the ready-for-use decolorizing agent, agents (A) and (B) are now mixed together. During the mixing process, the reducing agent, packaged separately beforehand, comes into contact with the oxidant and the two substances undergo an exothermic reaction with one another. The reaction heat generated by said exothermic reaction heats up the application mixture, and this process leads to a faster, better and complete dissolution of the reducing agent (e.g. the formamidine sulphinic acid) in the hydrous formulation.

A key and essential feature of the present disclosure is the ratio of reducing agent to oxidant in a total quantity of the agents (a) plus (b) (corresponding to the application mixture). In the total quantity of the agents (a) plus (b), the ratio of reducing agents to oxidants used is from about 50 to 1 to about 4 to 1. This allows the reducing agents to react with the oxidant. In this process, the oxidant is completely used up and the application mixture is heated up. As the application mixture is heated up, the residual reducing agent dissolves quickly and completely, and can therefore be effectively used for the reductive decoloration of dyed hair.

A first subject matter of the present disclosure is a kit-of-parts for the reductive decoloration of dyed keratinous fibers comprising, packaged separately from one another
(I) a first container (A) containing a cosmetic agent (a) and
(II) a second container (B) containing a cosmetic agent (b),
wherein
   the agent (a) in the first container (A)
(a1) contains one or more reducing agents from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid) (thioglycol acid) and/or ascorbic acid, and
   the agent (b) in container (B)
(b1) contains one or more oxidants from the group of hydrogen peroxide, potassium sulphate, sodium persulphate and/or ammonium persulphate, and
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

Keratinous fibers, keratin-containing fibers or keratin fibers are furs, wool, feathers and, in particular, human hair. Although the agents as contemplated herein are most suitable for lightening and coloring keratinous fibers and/or human hair, they can in principle be used for other purposes.

The expression “dyed keratinous fibers” means keratin fibers, which were dyed by employing conventional cosmetic dyes known to a person skilled in the art. The expression “dyed keratinous fibers” means in particular fibers that have been dyed by employing oxidative dyes and/or partially oxidizing dyes known from the prior art. In this context, explicit reference is made to the known monographies, e.g. Kh. Schrader, Grundlagen and Rezepturen der Kosmetika [Cosmetic principles and formulas], 2nd Edition, Huthig Buch Verlag, Heidelberg, 1989, which reflect the corresponding knowledge of a person skilled in the art.

Agent (a) in Container (A)

The kit-of-parts as contemplated herein comprises a first separately packaged container (A) with a cosmetic agent (a). The agent (a) is exemplified in that contains, as the ingredient (a1) essential to the present disclosure, at least one reducing agent from the group of formamidine sulphinic acid, sodium dithionite, zinc dithionite, potassium dithionite, hydroxymethane sulphinic acid, aminomethane sulphinic acid, cysteine, thiolactic acid, sulfanyl acetic acid (thioglycol acid) and/or ascorbic acid.

Formamidine sulphinic acid is also referred to as thiourea dioxide or aminoiminomethane sulphinic acid. Formamidine sulphinic acid has the structure of formula (I), and can also exist in the form of its tautomers. Formamidine sulphinic acid has the CAS Number 1758-73-2 and is commercially available from Sigma Aldrich, for example.

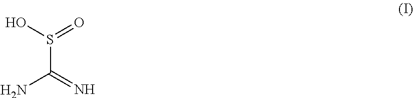

Sodium dithionite is an inorganic reducing agent and has the empirical formula Na₂S₂O₄ and CAS No. 7775-14-6.

Zinc dithionite is an inorganic reducing agent and has the empirical formula ZnS₂O₄ and CAS No. 7779-86-4.

Potassium dithionite is an inorganic reducing agent and has the empirical formula K₂S₂O₄ and CAS No. 14293-73-3.

Hydroxymethane sulphinic acid is an inorganic reducing agent and has the empirical formula HO—CH₂—S(O)OH and CAS No. 79-25-4. Hydroxymethane sulphinic acid is also referred to as formaldehyde sulfoxylic acid. As contemplated herein, both hydroxymethane sulphinic acid itself and the physiologically tolerated salts thereof, sodium salt and/or zinc salt, for example, can be used. Accordingly, the use of sodium formaldehyde sulfoxylate (sodium hydroxymethane sulphinic acid, the sodium salt of hydroxymethane sulphinic acid) and/or zinc formaldehyde sulfoxylate (zinc hydroxymethane sulphinate, the zinc salt of hydroxymethane sulphinic acid) is also as contemplated herein.

Amino methane sulphinic acid is an inorganic reducing agent and has the empirical formula H₂N—CH₂—S(O)OH and CAS No. 118201-33-5. As contemplated herein, both amino methane sulphinic acid itself and the physiologically tolerated salts thereof, sodium salt and/or zinc salt, for example, can be used. The use of sodium amino methane sulfinate (sodium salt of amino methane sulphinic acid) and/or zinc amino methane sulfinate (zinc salt of amino methane sulphinic acid) is therefore contemplated herein.

As contemplated herein, cysteine (2-amino-3-sulfanyl propionic acid) means D-cysteine, L-cysteine and/or a mixture of D- and L-cysteine.

Thio lactic acid (2-sulfanylpropionic acid) means D-thio-lactic acid, L-thio-lactic acid and/or a mixture of D- and L-thio lactic acid. The use of both thio lactic acid itself and also thio lactic acid in the form of a physiologically tolerable salt thereof are contemplated herein. A preferred salt of thio lactic acid is ammonium thiolactate.

Ammonium thiolactate is the ammonium salt of thio lactic acid (i.e. the ammonium salt of 2-sulfanylpropionic acid) (Formula XX).

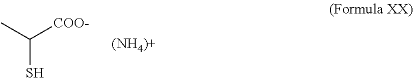

The definition of ammonium thiolactate includes both the ammonium salts of D-thio lactic acid, and also the ammonium salts of L-thio lactic acid and the mixtures thereof

Sulfanyl acetic acid (thioglycol acid, 2-mercapto acetic acid) is an organic reducing agent of the formula HS—CH₂—COOH, the compound has the CAS No. 68-11-1. In the case of thioglycol acid, both the use of thioglycol acid and the use of a physiologically tolerated salt of thioglycol acid is contemplated herein. Sodium thioglycolate, potassium thioglycolate and/or ammonium thioglycolate, for example, can be used as physiologically tolerated salts of thiolgycol acid. Ammonium thioglycolate is a preferred physiologically tolerated salt of thioglycol acid.

Ammonium thioglycolate is the ammonium salt of thioglycol acid (i.e. the ammonium salt of 2-sulfanyl acetic acid) (Formula XXX).

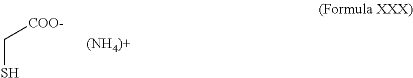

As contemplated herein, ascorbic acid means in particular (R)-5-[(S)-1,2-dihydroxyethyl]-3,4-dihydroxy-5H-furan-2-on (other alternative names: Vitamin C, L-ascorbic acid) with the CAS No. 50-81-7.

The reducing agents from the group of formamidine sulphinic acid, sodium dithionite, zinc dithionite and/or potassium dithionite have proven especially suitable for use in the kit as contemplated herein. The difficulties associated with dissolving formamidine sulphinic acid can be excellently avoided by the kit as contemplated herein. Therefore, the use of formamidine sulphinic acid is most preferred.

Therefore, most preferred is a kit-of-parts for the reductive decoloration of dyed keratinous fibers exemplified in that the agent (a) in the first container (A)
(a1) contains one or more reducing agents from the group of formamidine sulphinic acid, sodium dithionite, zinc dithionite and/or potassium dithionite, most preferably formamidine sulphinic acid.

Consequently, the preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) contains one or more reducing agents from the group of formamidine sulphinic acids, sodium dithionite, zinc dithionite, potassium dithionite, and
   the agent (b) in container (B)
(b1) contains one or more oxidants from the group of hydrogen peroxide, potassium sulphate, sodium persulphate and/or ammonium persulphate, and
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

The preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) Formamidine sulphinic acid, and
   the agent (b) in container (B)
(b1) contains one or more oxidants from the group of hydrogen peroxide, potassium sulphate, sodium persulphate and/or ammonium persulphate, and
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

The reducing agent(s) from the group (a1) are preferably used in the agent (a) in specific quantity ranges. Preferably, the agent (a) contains the reducing agent(s) in a total quantity from about 5.0 to about 80.0 wt. %, more preferably from about 10.0 to about 70.0 wt. %, even more preferably from about 20.0 to about 60.0 wt. % and most preferably from about 30.0 to about 50.0 wt. %. These data in percentage by weight are relative to the total weight of the agent (a).

Therefore, a kit-of-parts for the reductive decoloration of dyed keratinous fibers, exemplified in that the agent (a) in the first container (A)—contains relative to the total weight of the agent (a)—one or more reducing agents (a1) in a total quantity of from about 5.0 to about 80.0 wt. %, preferably from about 10.0 to about 70.0 wt. %, more preferably from about 20.0 to about 60.0 wt. % and most preferably from about 30.0 to about 50.0 wt. %, is most preferred.

Agent (a) is an agent, containing the reducing agents, which has to meet especially high requirements with respect to storage stability. The unwanted and premature decomposition of the reducing agent is more severe in hydrous solutions. Therefore, it is most preferable for the agent (a) to be packaged in a substantially anhydrous manner. “Substantially anhydrous” means that the water content of the agent is (a) is maximum about 10.0 wt. %. Certain water quantities can be entrained in the agent, for example, if a raw material in the form of a hydrate or a solution is used. However, the water content of the agent (a) is preferably below about 10.0 wt. %, more preferably below about 5.0 wt. %, even more preferably below about 2.5 wt. % and most preferably below about 0.1 wt. %. All percentage by weight data are relative to the total weight of the agent (a).

Therefore, a kit-of-parts for the reductive decoloration of dyed keratinous fibers, exemplified in that the agent (a) in the first container (A)—has relative to the total weight of the agent (a)—a water content of below about 10.0 wt. %, preferably below about 5.0 wt. %, more preferably below about 2.5 wt. % and most preferably below about 0.1 wt. %, is most preferred.

The substantially anhydrous agent (a) can be, for example, a powder or even a paste. If the agent (a) is used in the form of a powder, dust formation must be avoided and/or the powder must be dedusted. Therefore, it is especially advantageous for the agent (a) to exist in the form of a paste.

To obtain a pasty agent (a), the reducing agent(s) can be worked into a fatty carrier, for example. The consistency of the paste is partially determined by the melting point of the fatty constituents.

Therefore, most preferred is a kit-of-parts for the reductive decoloration of dyed keratinous fibers exemplified in that the agent (a) in the first container (A)
(a2) contains one or more fatty constituents (a2) from the group of C₁₂-C₃₀ fatty alcohols, C₁₂-C₃₀ fatty acid triglycerides, C₁₂-C₃₀fatty acid monoglycerides, C₁₂-C₃₀ fatty acid diglycerides, C₁₂-C₃₀ fatty acid esters, hydrocarbons and/or silicone oils.

It is most preferred for the agent (a) to contain as the constituents (a2) one or more fatty constituents from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, hydrocarbons and/or silicone oils.

To the extent required by the present disclosure, “fatty constituents” are organic compounds with a water solubility at room temperature (22° C.) and atmospheric pressure (760 mmHg) of less than about 1 wt. %, preferably less than about 0.1 wt.-%.

The definition of fatty constituents explicitly includes only uncharged (i.e. Non-ionic) compounds. Fatty constituents have at least one saturated or unsaturated alkyl group with at least about 12 carbon atoms. The molecular weight of the fatty constituents is a maximum about 5000 g/mol, preferably maximum about 2500 g/mol and even more preferably a maximum of about 1000 g/mol. The fatty constituents are neither polyoxyalkylated nor polyglycerylated compounds. In this context, polyalkoxylated compounds are those compounds made using at least 2 alkylenoxide units. Likewise, polyglycerized compounds are those compounds made using at least two glycerine units.

Because according to the present disclosure, only non-ionic substances are explicitly considered as fatty constituents, charged compounds such as fatty acids and the salts thereof do not fall into the group of fatty constituents.

Referred fatty constituents are the constituents from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters and hydrocarbons.

The C₁₂-C₃₀ fatty alcohols can be saturated, one or more unsaturated, linear or branched fatty alcohols with about 12 to about 30 C-atoms.

Examples of preferred linear, saturated C₁₂-C₃₀ fatty alcohols are dodecan-1-ol (dodecylalcohol, laurylalcohol), tetradecan-1-ol (retradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear, unsaturated fatty alcohols are (9Z)-octadec-9-en-1-ol (oleyl alcohol), (9E)-octadec-9-en-1-ol (elaidyl alcohol), (9Z,12Z)-cctadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidic alcohol ((5Z,8Z,11Z,14Z)-eicosa-5,8,11,14-tetraen-1-ol), erucyl alcohol ((13Z)-docos-13-en-1-ol) and/or brassidyl alcohol ((13E)-docosen-1-ol).

The preferred typical branched fatty alcohols are 2-octyl-dodecanol, 2-hexyl-dodecanol and/or 2-butyl-dodecanol.

To the extent required by the present disclosure, a C₁₂-C₃₀ fatty acid triglyceride is the triester of the trivalent alcohol glycerine with three equivalent fatty acids. Both identically structured and different fatty acids within a triglyceride molecule can be involved in the ester formation.

To the extent required by the present disclosure, fatty acids are saturated or unsaturated, unbranched or branched, unsubstituted or substituted C₁₂-C₃₀carboxylic acids. Unsaturated fatty acids can be unsaturated or polyunsaturated. The C—C double bond(s) of an unsaturated fatty acid can have the cis- or trans configuration.

Fatty acid triglycerides are exemplified by their particular suitability, for which at least one of the ester groups, based on glycerine, is formed with a fatty acid, which is selected from dodecan acid (laurin acid), tetradecan acid (myristine acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid [(9Z)-octadec-9-enic acid], elaidic acid [(9E)-octadec-9-enic acid], erucic acid [(13Z)-docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid].

The fatty acid triglycerides can also be from natural sources. The fatty acid triglycerides occurring in soy bean oil, peanut oil, sunflower oil, macadamia nut oil, drumstick tree oil, apricot kernel oil, manila oil and/or possibly hardened castor oil, and the mixtures thereof are particularly suitable for use in the agent (a) as contemplated herein.

A C₁₂-C₃₀ fatty acid monoglyceride is the monoester of the trivalent alcohol glycerine with an equivalent fatty acid. Either the middle hydroxy group of the glycerine or the final hydroxy group of the glycerin can be esterified with the fatty acid.

The C₁₂-C₃₀ fatty acid triglycerides are exemplified by their particular suitability, for which at least one hydroxy group of the glycerine is esterified, wherein the fatty acids are selected from dodecan acid (laurin acid), tetradecan acid (myristine acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-Hexadec-9-enic acid], oleic acid [(9Z)-octadec-9-enic acid], elaidic acid [(9E)-octadec-9-enic acid], erucic acid [(13Z)-Docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] or nervonic acid [(15Z)-Tetracos-15-enic acid].

A C₁₂-C₃₀ fatty acid diglyceride is the diester of the trivalent alcohol glycerine with two equivalent fatty acids. Either the middle or an independent hydroxy group of the glycerine with two equivalent fatty can be esterified with two equivalent fatty acids or both final hydroxy groups of the glycerin are each esterified with one fatty acid. The glycerin can be esterified with two identically structured or two different fatty acids.

Fatty acid diglycerides are exemplified by their particular suitability, for which at least one of the ester groups, based on glycerine, is formed with a fatty acid, which is selected from dodecan acid (laurin acid), tetradecan acid (myristine acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid [(9Z)-octadec-9-enic acid], elaidic acid [(9E)-octadec-9-enic acid], erucic acid [(13Z)-docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid].

A C₁₂-C₃₀ fatty acid ester according to the present disclosure is the monoester from a fatty acid and an aliphatic, monovalent alcohol, wherein the alcohol comprises up to about 6 carbon atoms. Suitable alcohols include ethanol, n-propanol, isopropanol, 1-butanol, Isobutanol, tert-butanol, n-pentanol, iso-pentanol or n-hexanol. Preferred alcohols are ethanol and isopropanol.

Preferred C₁₂-C₃ fatty acid esters are the esters formed from the esterification of the alcohols ethanol and/or isopropanol with one of the fatty acids from the group of dodecan acid (lauric acid), tetradecan acid (myristine acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid [(9Z)-octadec-9-enic acid], elaidic acid [(9E)-octadec-9-enic acid], erucic acid [(13Z)-docos-13-enic acid], linoleic acid [(9Z, 12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid]. Of the fatty acid esters, isopropylmyristate is most preferred.

Hydrocarbons are exclusively compounds comprising the atoms hydrocarbon and hydrogen having from about 8 to about 250 carbon atoms, preferably from about 8 to about 150 carbon atoms. In this context, aliphatic hydrocarbons such as mineral oils, liquid paraffin oils (e.g. paraffinum liquidum or paraffinum perliquidum), isoparaffin oils, semi-solid paraffin oils, paraffin waxes, hard paraffin (paraffinum solidum), vaseline and polydecene are preferred.

Liquid paraffin oils (paraffinum liquidum and paraffinum perliquidum) have proven particularly suitable. The most preferred hydrocarbon is paraffinum liquidum, also referred to as white oil. Paraffinum liquidum is a mixture of cleaned, saturated, aliphatic hydrocarbons, which includes mainly of hydrogen chains with a C-chain distribution from about 25 to about 35 C-atoms.

The fatty constituents can be the cosmetic carrier of the agent (a) and also have—depending on the nature and quantity of the fat used—a significant influence on the consistency of the agent. In this context, it has proven most preferable for the agent (a) to contain one or more fatty constituents in a total quantity from about 10 to about 90 wt. %, preferably from about 20 to about 60 wt. % and most preferably from about 25 to about 50 wt. %, said quantity values being relative to the total weight of the agent (a).

Therefore, it is most preferable for a kit-of-parts for the reductive decoloration of dyed keratinous fibers, exemplified in that the agent (a) in the first container (A)—to contain relative to the total weight of the agent (a)—one or more fatty constituents (a2) from the group of C12-C30 fatty alcohols, C12-C30 fatty acid triglycerides, C12-C30fatty acid monoglycerides, C12-C30 fatty acid diglycerides, C12-C30 fatty acid esters, hydrocarbons and/or silicone oils in a total quantity of from about 10 to about 90 wt. %, preferably from about 20 to about 60 wt. % and most preferably from about 25 to about 50 wt. %.

The use of hydrocarbons has proven to be particularly effective for reducing dust formation and rendering the reducing agent inert to atmospheric oxygen. Paraffin oils and paraffin wax in particular have proven to be highly compatible with the solid, inorganic reducing agents. For this reason, the use of one or more hydrocarbons in a total quantity from about 15.0 to about 90.0 wt. %, preferably from about 20.0 to about 85.0 wt. %, more preferably from about 25.0 to about 80.0 wt. % and most preferably from about 30.0 to about 75.0 wt. %—relative to the total weight of the agent (a)—as the reducing agent (a2) is explicitly most preferred.

Moreover, the preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) contains one or more reducing agents from the group of formamidine sulphinic acids, sodium dithionite, zinc dithionite, potassium dithionite, and
(a2) one or more fatty constituents (a2) from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, hydrocarbons and/or silicone oils, and
   the agent (b) in container (B)
(b1) contains one or more oxidants from the group of hydrogen peroxide, potassium sulphate, sodium persulphate and/or ammonium persulphate, and
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

Moreover, the preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) contains formamidine sulphinic acid and
(a2) paraffin oil and
   the agent (b) in container (B)
(b1) contains one or more oxidants from the group of hydrogen peroxide, potassium sulphate, sodium persulphate and/or ammonium persulphate, and
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

Moreover, the preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) contains formamidine sulphinic acid and
(a2) paraffin oil and
   the agent (b) in container (B)
(b1) contains hydrogen peroxide, and
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

Various reducing agents achieve their optimum effect within a specific pH value range. Ready-to-use decoloration agent with dithionite salts are preferably set to an acid pH value, whereas ready-to-use decoloration agents with formamidine sulphinic acid achieve their best effect in an alkali environment.

As already described, the ready-to-use decoloration agent is produced shortly before application by mixing the agents (a) and (b). The agent (b) contains one or more oxidants. Particularly when using hydrogen peroxide, it is advisable to set the agent (b) to an acid pH value, since hydrogen peroxide decomposes in alkali solutions.

If the pH value of the ready-to-use agent is supposed to be set within the alkali range, working the required alkalizing agent into the agent (a) proves advantageous. As already described, agent (a) is preferably a powder or a paste. Therefore, the use of an alkalizing agent in the agent (a) is particularly preferred.

Suitable alkalizing agents include sodium metasilicate, potassium metasilicate, potassium hydroxide, sodium hydroxide, sodium carbonate, potassium carbonate, sodium hydrogen carbonate and/or potassium hydrogen carbonate

Therefore, most preferred is a kit-of-parts for the reductive decoloration of dyed keratinous fibers exemplified in that the agent (a) in the first container (A)
(a3) contains one or more alkalizing agents from the group of sodium metasilicate, potassium metasilicate, potassium hydroxide, sodium hydroxide, sodium carbonate, potassium carbonate, sodium hydrogen carbonate and/or potassium hydrogen carbonate.

Moreover, the preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) contains one or more reducing agents from the group of formamidine sulphinic acids, sodium dithionite, zinc dithionite, potassium dithionite, and
(a2) one or more fatty constituents (a2) from the group of C12-C30 fatty alcohols, C12-C30 fatty acid triglycerides, C12-C30 fatty acid monoglycerides, C12-C30 fatty acid diglycerides, C12-C30 fatty acid esters, hydrocarbons and/or silicone oils, and
(a3) contains one or more alkalizing agents from the group of sodium metasilicate, potassium metasilicate, potassium hydroxide, sodium hydroxide, sodium carbonate, potassium carbonate, sodium hydrogen carbonate and/or potassium hydrogen carbonate, and
   the agent (b) in container (B)
(b1) contains one or more oxidants from the group of hydrogen peroxide, potassium sulphate, sodium persulphate and/or ammonium persulphate,
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

The quantity of alkalizing agent (a3), which is used in the agent (a), determines a pH value for the application mixture, and also influences the exothermic reaction between reducing agent and oxidant. In this context, the best results can be achieved if one of more alkalizing agents is used in the agent (a) in a total quantity from about 3.0 to about 25.0 wt. %, preferably from about 5.0 to about 21.0 wt. %, more preferably from about 7.0 to about 17 wt. % and most preferably from about 9.0 to about 13.0 wt. %. The percentage by weight data of the alkalizing agent are relative to the total weight of the agent (a).

Within the group of solid alkalizing agents (a3), the use of sodium silicate has proven particularly suitable.

Moreover, the preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) contains formamidine sulphinic acid and
(a2) paraffin oil and
(a3) sodium metasilicate
   the agent (b) in container (B)
(b1) contains one or more oxidants from the group of hydrogen peroxide, potassium sulphate, sodium persulphate and/or ammonium persulphate, and
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

Therefore, a kit-of-parts for the reductive decoloration of dyed keratinous fibers, exemplified in that the agent (a)—relative to the total weight of the agent (a)—contains from about 3.0 to about 25.0 wt. %, preferably from about 5.0 to about 21.0 wt. %, more preferably from about 7.0 to about 17 wt. % and most preferably from about 9.0 to about 13.0 wt. % sodium metasilicate, is most preferred.

Agent (b) in Container (B)

The second container (B) of the kit-of-parts as contemplated herein contains the second cosmetic agent (b). This is exemplified in that it contains one or more oxidants from the group of hydrogen peroxide, potassium sulphate, sodium persulphate and/or ammonium persulphate,

Preferably, the agent (b) is packaged in a hydrous or anhydrous manner. The cosmetic agent (b) can be, for example, an agent having a suitable hydrous or hydrous-alcoholic carrier. Carriers such as creams, emulsions, gels or tenside-containing, foaming solutions, such as shampoos, foaming aerosols, foam formulations or other preparations suitable for application on the hair, are used for the purpose of reductive decoloration. Agents for the reductive decoloration of keratinous fibers are preferably creams, emulsions or free-flowing gels. Most preferably, the agent (b) is formulated as an emulsion.

Hydrogen peroxide is most preferably used as the oxidant in the agent (b). Hydrogen peroxide in the form of a hydrous solution is especially suitable. If the reducing agents of the agent (a) come into contact with hydrogen peroxide in the agent (b), the exothermic reaction that occurs is particularly controllable and foreseeable.

Therefore, most preferred is a kit-of-parts for the reductive decoloration of dyed keratinous fibers exemplified in that the agent (b) in the first container (B) includes
(b1) hydrogen peroxide as the oxidant.

Consequently, the preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) contains one or more reducing agents from the group of formamidine sulphinic acids, sodium dithionite, zinc dithionite, potassium dithionite, and
   the agent (b) in container (B)
(b1) contains hydrogen peroxide,
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

The preference is for a kit-of-parts for the reductive decoloration of dyed hair comprising, packaged separately from one another
(I) a first container (A) containing a cosmetic agent (a) and
(II) a second container (B) containing a cosmetic agent (b),
wherein
   the agent (a) in the first container (A)
(a1) contains formamidine sulphinic acid, and
   the agent (b) in container (B)
(b1) contains hydrogen peroxide,
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

The quantity of oxidant in the agent (B) is especially important. Preferably, the agent (b) in the container (B)—contains relative to the total weight of the agent (b)—one or more oxidants (b1) in a total quantity of from about 1.0 to about 5.0 wt. %, preferably from about 0.15 to about 2.5 wt. %, more preferably from about 0.2 to about 1.0 wt. % and most preferably from about 0.25 to about 0.8 wt. %.

Accordingly, it is preferable for the agent (b) in container (B) to contain—relative to the total weight of the agent (b)—from about 0.15 to about 2.5 wt. %, more preferably from about 0.2 to about 1.0 wt. % and most preferably from about 0.25 to about 0.8 wt. % hydrogen peroxide.

Therefore, a kit-of-parts for the reductive decoloration of dyed keratinous fibers, exemplified in that the agent (b) in the second container (B)—contains relative to the total weight of the agent (b)—one or more oxidants agents (b1) in a total quantity of from about 0.1 to about 5.0 wt. %, preferably from about 0.15 to about 2.5 wt. %, more preferably from about 02.0 to about 1.0 wt. % and most preferably from 0.25 to 08.0 wt. %, is most preferred. The agent (b) contains oxidants, most preferably hydrogen peroxide. To stabilize the oxidant, these agents are preferably set to an acid pH from about 2 to about 7, preferably from about 2 to about 5.

The pH values can, for example, be measured with a Type N61 glass electrode from Schott at a temperature of 22° C. To set the acid pH value, the agent (b) preferably contains one or more organic and/or inorganic acids.

To set the pH value, one or more acids from the group of citric acid, tartaric acid, malic acid, lactic acid, acetic acid, sulphuric acid, hydrochloric acid, phosphoric acid, methane sulfonic acid, benzoic acid, malonic acid, oxalic acid, and/or 1-hydroxyethane-1,1-diphosphonic acid, have proven particularly suitable. Preferably, the acids are selected from the group of citric acid, tartaric acid, malic acid, lactic acid, methane sulfonic acid, benzoic acid, hydrochloric acid, sulphuric acid, phosphoric acid and/or 1-hydroxyethane-1,1-diphosphonic acid.

Moreover, the preference is for a kit-of-parts for the reductive decoloration of dyed keratinous fibers comprising, packaged separately from one another
(I) a first container (A) containing a cosmetic agent (a) and
(II) a second container (B) containing a cosmetic agent (b),
wherein
   the agent (a) in the first container (A)—relative to the total weight of the agent (a)
(a1) contains one or more reducing agents from the group of formamidine sulphinic acids, sodium dithionite, zinc dithionite, potassium dithionite, hydroxymethane sulphinic acid, aminomethane sulphinic acid, cysteine, thio lactic acid, sulfanyl acetic acid (thioglycol acid) and/or ascorbic acid in a total quantity from about 5.0 to about 80.0 wt. %, and
   the agent (b) in the container (B)—relative to the total weight of the agent (b)
(b1) contains on or more oxidants from the group of hydrogen peroxide, potassium sulphate, sodium sulphate and/or ammonium persulfate in a total quantity from about 0.1 to about 5.0 wt. %,
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

Moreover, the preference is for a kit-of-parts for the reductive decoloration of dyed keratinous fibers comprising, packaged separately from one another
(I) a first container (A) containing a cosmetic agent (a) and
(II) a second container (B) containing a cosmetic agent (b),
wherein
   the agent (a) in the first container (A)—relative to the total weight of the agent (a)
(a1) contains one or more reducing agents from the group of formamidine sulphinic acids, sodium dithionite, zinc dithionite, potassium dithionite, hydroxymethane sulphinic acid, aminomethane sulphinic acid, cysteine, thio lactic acid, sulfanyl acetic acid (thioglycol acid) and/or ascorbic acid in a total quantity from about 10.0 to about 70.0 wt. %, and
   the agent (b) in the container (B)—relative to the total weight of the agent (b)
(b1) contains on or more oxidants from the group of hydrogen peroxide, potassium sulphate, sodium sulphate and/or ammonium persulfate in a total quantity from about 0.15 to about 2.5 wt. %,
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

Moreover, the preference is for a kit-of-parts for the reductive decoloration of dyed keratinous fibers comprising, packaged separately from one another
(I) a first container (A) containing a cosmetic agent (a) and
(II) a second container (B) containing a cosmetic agent (b),
wherein
   the agent (a) in the first container (A)—relative to the total weight of the agent (a)
(a1) contains one or more reducing agents from the group of formamidine sulphinic acids, sodium dithionite, zinc dithionite, potassium dithionite, hydroxymethane sulphinic acid, aminomethane sulphinic acid, cysteine, thio lactic acid, sulfanyl acetic acid (thioglycol acid) and/or ascorbic acid in a total quantity from about 2.0 to about 60.0 wt. %, and
   the agent (b) in the container (B)—relative to the total weight of the agent (b)
(b1) contains on or more oxidants from the group of hydrogen peroxide, potassium sulphate, sodium sulphate and/or ammonium persulfate in a total quantity from about 0.2 to about 1.0 wt. %,
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about4.0.

Moreover, the preference is for a kit-of-parts for the reductive decoloration of dyed keratinous fibers comprising, packaged separately from one another
(I) a first container (A) containing a cosmetic agent (a) and
(II) a second container (B) containing a cosmetic agent (b),
wherein
   the agent (a) in the first container (A)—relative to the total weight of the agent (a)
(a1) contains one or more reducing agents from the group of formamidine sulphinic acids, sodium dithionite, zinc dithionite, potassium dithionite, hydroxymethane sulphinic acid, aminomethane sulphinic acid, cysteine, thio lactic acid, sulfanyl acetic acid (thioglycol acid) and/or ascorbic acid in a total quantity from about 30.0 to about 50.0 wt. %, and
   the agent (b) in the container (B)—relative to the total weight of the agent (b)
(b1) contains on or more oxidants from the group of hydrogen peroxide, potassium sulphate, sodium sulphate and/or ammonium persulfate in a total quantity from about 0.25 to about 0.8 wt. %,
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

As already described, the agent (b) in container (B) is most preferably a hydrous cosmetic carrier formulation. The water content—relative to the total weight of the agent (b) is preferably from about 30 to about 97 wt. %, more preferably from about 40 to about 95, even more preferably from about 50 to about 93 and most preferably from about 60 to about 91 wt. %.

Therefore, a kit-of-parts for the reductive decoloration of dyed keratinous fibers, exemplified in that the agent (b)—relative to the total weight of the agent (b)—contains from about 30 to about 97 wt. %, preferably from about 40 to about 95, more preferably from about 50 to about 93 and most preferably from about 60 to about 91 wt. % of water, is most preferred.

Moreover, the preference is for a kit-of-parts for the reductive decoloration of dyed keratinous fibers comprising, packaged separately from one another
(I) a first container (A) containing a substantially anhydrous cosmetic agent (a) and
(II) a second container (B) containing a hydrous cosmetic agent (b),
wherein
   the agent (a) in the first container (A)
(a1) contains one or more reducing agents from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid) (thioglycol acid) and/or ascorbic acid, and
   the agent (b) in container (B)
(b1) contains one or more oxidants from the group of hydrogen peroxide, potassium sulphate, sodium persulphate and/or ammonium persulphate,
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0

Mixture of Agents (a) Plus (b)
To produce the ready-to-use decolorizing agent, the agents (a) and (b) are mixed with one another, the intention being to mix the entire quantity of the agent (a) in container (A) with the total quantity of the agent (b) in container (B). In other words, the total quantity of agent (a) plus (b) constitutes the ready-to-use decolorizing agent.
The mixing can be achieved by transferring the total content from container (A) to container (B) (in this case container (B) is larger than container (A)). Transferring the total content from container (B) to container (A) (in this case a correspondingly larger container (A) is selected) is also contemplated herein.

When the agents (a) and (b) are mixed, an exothermic reaction now occurs between the reducing agents (a1), which were previously contained in the agent (a), and the oxidants (b1), which were previously contained in the agent (b), now occurs.

Because the extent of the exothermic reaction depends on the quantities of the reducing agents (a1) and oxidants (b1) in the ready-to-use agent (agents (a) plus agent (b), all values for the weight ratio (a1)/(b1) are also relative to the total weight of the agents (a) plus (b).

A key and essential feature of the kit as contemplated herein is that the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0

### EXAMPLE 1

Container (A) contains 20 g of the agent (a). Agent (a) contains (a1) 8.0 g of formamidine sulphinic acid.
The agent (a) contains—relative to the total weight of the agent (a)—40 wt. % of formamidine sulphinic acid (a1) (8.0 g/20 g=40 wt. %).
Container (B) contains 80 g of the agent (b). The agent (b) contains (b1) 0.4 g of hydrogen peroxide.
The agent (b) contains—relative to the total weight of the agent (b)—0.5 wt. % hydrogen peroxide.
To produce the ready-to-use decolorizing agent, 20 g of the agent (a) are mixed with 80 g of the agent (b) (total weight of agent (a) plus (b)=100 g).
The total quantity of the agents (a)+(b) (corresponding to the ready-to-use decolorizing agent) contain:
(a1) 8.0 g formamidine sulphinic acid (8 wt. % relative to agents (a) plus (b))
(a1) 0.4 g hydrogen peroxide (0.4 wt. % relative to agents (a) plus (b))
The total weight ratio of the total quantity of all reducing agents (a1) contained in agent (a) to the total quantity of all oxidants (b1) contained in agent (b)—relative to the total weight of agents (a) plus (b)—is (8.0 g/0.4 g=20).

### EXAMPLE 2

Container (A) contains 50 g of the agent (a). Agent (a) contains (a1) 8.0 g of formamidine sulphinic acid.
The agent (a) contains—relative to the total weight of the agent (a)—16 wt. % of formamidine sulphinic acid (a1) (8.0 g/50 g=16 wt. %).
Container (B) contains 80 g of the agent (b). The agent (b) contains (b1) 0.8 g of hydrogen peroxide.
The agent (b) contains—relative to the total weight of the agent (b)—1.0 wt. % hydrogen peroxide.
To produce the ready-to-use decolorizing agent, 50 g of the agent (a) are mixed with 80 g of the agent (b) (total weight of agent (a) plus (b)=130 g).
The total quantity of the agents (a)+(b) (corresponding to the ready-to-use decolorizing agent) contain:
(a1) 8.0 g formamidine sulphinic acid (6.2 wt. % relative to agents (a) plus (b))
(a1) 0.8 g hydrogen peroxide (0.62 wt. % relative to agents (a) plus (b))
The total weight ratio of the total quantity of all reducing agents (a1) contained in agent (a) to the total quantity of all oxidants (b1) contained in agent (b)—relative to the total weight of agents (a) plus (b)—is (8.0 g/0.8 g=10).

### EXAMPLE 3

Container (A) contains 100 g of the agent (a). Agent (a) contains (a1) 5.0 g of formamidine sulphinic acid.
The agent (a) contains—relative to the total weight of the agent (a)—5 wt. % of formamidine sulphinic acid (a1) (5.0 g/100 g=5 wt. %).
Container (B) contains 100 g of the agent (b). The agent (b) contains (b1) 1.0 g of hydrogen peroxide.
The agent (b) contains—relative to the total weight of the agent (b)—1.0 wt. % hydrogen peroxide.
To produce the ready-to-use decolorizing agent, 100 g of the agent (a) are mixed with 100 g of the agent (b) (total weight of agent (a) plus (b)=200 g).
The total quantity of the agents (a)+(b) (corresponding to the ready-to-use decolorizing agent) contain:
(a1) 5.0 g formamidine sulphinic acid (2.5 wt. % relative to agents (a) plus (b))
(b1) 1.0 g hydrogen peroxide (0.5 wt. % relative to agents (a) plus (b))
The total weight ratio of the total quantity of all reducing agents (a1) contained in agent (a) to the total quantity of all oxidants (b1) contained in agent (b)—relative to the total weight of agents (a) plus (b)—is (5.0 g/1.0 g=5).

Accordingly, in the total quantity of the agents (a) plus (b), the ratio of reducing agents (a1) to oxidants (b1) used is from about 4 to about 50 to 1. This surplus guarantees that after the reaction between reducing agent and oxidant, there is still sufficient reducing agent remaining to reductively decolor the dyed hair. The greater the surplus, the more substance available for reductive decoloration. Conversely, completely dissolving a large quantity of reducing agent is also difficult. For these reasons, the weight ratio (a1)/(b1) is preferably set to specific ranges of between from about 45.0 to about 5.0, more preferably from about 40.0 to about 8.0, even more preferably from about 30.0 to about 12.0 and most preferably from about 25.0 to about 15.0.

### EXAMPLE

The weight ratio of the total quantity of all reducing agents (a1) contained in agent (a) to the total quantity of all oxidants (b1) contained in agent (b)—relative to the total weight of agents (a) plus (b)—is a value of from about 25.0 to about 15.0. In the total quantity of the agents (a) plus (b), from about 15 to about 25 times more reducing agent (a1) (measured in g) than oxidant (b1) (measured in g) is used.

Therefore, most preferable is a kit-of-parts for the reductive decoloration of dyed keratinous fibers, exemplified in that the weight ratio from the total quantity of all reducing agents (a1) contained in the agent (a) to the total quantity of all oxidants (b1) contained in the agent (b)—relative to the total weight of the agent (a) plus (b)—is a value of from about 45.0 to about 5.0, preferably from about 40.0 to about 8.0, more preferably from about 30.0 to about 12.0 and most preferably from about 25.0 to about 15.0.

As the aforementioned examples show, the total weight ratio of the total quantity of all reducing agents (a1) contained in agent (a) to the total quantity of all oxidants (b1) contained in agent (b)—relative to the total weight of agents (a) plus (b)—is determined by 4 factors.
(1) Quantity of reducing agent (a1), which is used in the agent (a)
(2) Quantity of the agent (a) in the container (A)
(2) Quantity of oxidant (b1), which is used in the agent (b)
(3) Quantity of the agent (b) in the container (B)

Depending on the desired application conditions, a person skilled in the art is able to set these 4 factors accordingly (always on the proviso that the ratio condition (a1)/(b1) remains fulfilled).

If a very fast and strong local heat development is desired, it is advantageous to provide a smaller quantity of the agent (a), wherein the agent (a) contains the reducing agent (a1) in a concentrated form—for example:
Container (A) contains 10 g of the agent (a). Agent (a) contains (a1) 8.0 g of formamidine sulphinic acid.
The agent (a) contains—relative to the total weight of the agent (a)—80 wt. % of formamidine sulphinic acid (a1) (8.0 g/10 g=80 wt. %).
Container (B) contains 100 g of the agent (b). The agent (b) contains (b1) 0.3 g of hydrogen peroxide.
The agent (b) contains—relative to the total weight of the agent (b)—0.3 wt. % hydrogen peroxide.

To produce the ready-to-use decolorizing agent, 10 g of the agent (a) are mixed with 100 g of the agent (b) (total weight of agent (a) plus (b)=110 g).
The total quantity of the agents (a)+(b) (corresponding to the ready-to-use decolorizing agent in the method) contain:
(a1) 8.0 g formamidine sulphinic acid (7.3 wt. % relative to agents (a) plus (b))
(b1) 0.3 g hydrogen peroxide (0.27 wt. % relative to agents (a) plus (b))
The total weight ratio of the total quantity of all reducing agents (a1) contained in agent (a) to the total quantity of all oxidants (b1) contained in agent (b)—relative to the total weight of agents (a) plus (b)—is (8.0 g/0.3 g=27).

Conversely, the exothermic reaction is easier to control if a larger quantity of the agent (a) is provided in the kit, wherein the agent (a) contains the reducing agent (a1) in a less concentrated form—for example:
Container (A) contains 20 g of the agent (a). Agent (a) contains (a1) 4.0 g of formamidine sulphinic acid.
The agent (a) contains—relative to the total weight of the agent (a)—20 wt. % of formamidine sulphinic acid (a1) (4.0 g/20 g=20 wt. %).
Container (B) contains 90 g of the agent (b). The agent (b) contains (b1) 0.15 g of hydrogen peroxide.
The agent (b) contains—relative to the total weight of the agent (b)—0.16 wt. % hydrogen peroxide.

To produce the ready-to-use decolorizing agent, 20 g of the agent (a) are mixed with 90 g of the agent (b) (total weight of agent (a) plus (b)=110 g).
The total quantity of the agents (a)+(b) (corresponding to the ready-to-use decolorizing agent in the method) contain:
(a1) 4.0 g formamidine sulphinic acid (3.6 wt. % relative to agents (a) plus (b))
(b1) 0.15 g hydrogen peroxide (0.14 wt. % relative to agents (a) plus (b))
The total weight ratio of the total quantity of all reducing agents (a1) contained in agent (a) to the total quantity of all oxidants (b1) contained in agent (b)—relative to the total weight of agents (a) plus (b)—is (4.0 g/0.15 g=27).

Containers (A) and (B) of the kit-of-parts as contemplated herein can contain various quantities of the agents (a) and (b).

In a suitable embodiment, the agents (a) and (b) are mixed in a quantity ratio of 1:1 (e.g. 100 g agent (a) and 100 g agent (b)) (always on the proviso that the ratio condition (a1)/(b1) remains fulfilled).

To guarantee the fullest possible dissolution of the reducing agent, however, it is advantageous to provide a surplus of the agent (b) over the agent (a). Therefore, it is particularly advantageous for the containers (A) and (B) to contain the agents (a) and (b) in such quantities that the quantity ratio of agent (a) to agent (b), i.e. the quantity ratio (a)/(b), is a value of from about 0.1 to about 1.0, preferably from about 0.15 to about 0.9, more preferably from about 0.18 to about 0.8 and most preferably from about 0.2 to about 0.5.

### EXAMPLE

Container (A) contains 10 g of the agent (a)
Container (B) contains 100 g of the agent (b)
The quantity ratio of the agent (a) to the agent (b), i.e. the quantity ratio (a)/(b), is a value of 10g/100g=0.1

### EXAMPLE

Container (A) contains 20 g of the agent (a)
Container (B) contains 80 g of the agent (b)
The quantity ratio of the agent (a) to the agent (b), i.e. the quantity ratio (a)/(b), is a value of 20g/80g=0.25

Therefore, most preferable is a kit-of-parts for the reductive decoloration of dyed keratinous fibers, which is exemplified in that the containers (A) and (B) contain the agents (a) and (b) in such quantities that the quantity ratio of agent (a) to agent (b), i.e. the quantity ratio (a)/(b), is a value of from about 0.1 to about 1.0, preferably from about 0.15 to about 0.9, more preferably from from about 0.18 to about 0.8 and most preferably from about 0.2 to about 0.5.

In other words, it is most preferable for the containers (A) and (B) to contain, in the kit-of-parts as contemplated herein, the agents (a) and (b) in such quantities that the agent (b) is present with a one to ten times surplus, preferably from about a 2 to about 5 times surplus, over the agent (a).

As contemplated herein, the agent (a) is free of oxidants. Further, as contemplated herein, the agent (b) is free of reducing agents.

### 2-Component System

The kit-of-parts as contemplated herein is a kit comprising a container (A) and a container (B). In principle, this kit can also comprise other containers, for example a container (C) containing a conditioning agent, a shampoo and a post-treatment agent.

However, it is most preferable for the kit-of-parts to comprise precisely two containers (A) and (B), i.e. the kit contains the agents (a) and (b), but no further separately packaged agents. If, within this embodiment, the addition of nourishing, cleansing or conditioning constituents is desired, such agents can be incorporated either into the agent (a), into the agent (b), or into both agents (a) and (b).

Therefore, most preferred is a kit-of-parts for the reductive decoloration of dyed keratinous fibers exemplified in that the kit comprises precisely two agents (a) and (b) in the two containers (A) and (B).

Further Constituents in the Agents (a) and/or (b)

The agents (a) and/or (b) can also contain further constituents and/or active ingredients. For example, the use of non-ionic tensides in the agents (a) and/or (b) has proven to be particularly advantageous.

Tensides include amphiphilic (bi-functional) compounds having at least one hydrophobic radical and at least one hydrophilic molecule. The hydrophilic molecule is usually a hydrocarbon chain having from about 10 to about 30 carbon atoms. In the case of non-ionic tensides, the hydrophilic molecule comprises an uncharged, highly-polar structural unit.

Nonionic tensides include, for example, at least one polyol group, a polyalkylene glycol ether group or a combination of a polyol and polyglycol ether group. Examples of such compounds include

Adducts of from about 2 to about 50 moles ethylene oxide and/or from about 2 to about 50 moles propylene oxide on linear and branched fatty alcohols having from about 12 to about 30 carbon atoms and/or the fatty alcohol polypropylene glycolethers and/or mixed fatty alcohol polyethers,

adducts of from about2 to about 50 moles ethylene oxide and/or from about 2 to about 50 moles propylene oxide on linear and branched fatty acids having from about 6 to about 30 carbon atoms, the fatty acid polypropylene glycolethers and/or the fatty acid polypropylene glycolethers and/or mixed fatty acid polyethers,

adducts of from about 2 to about 50 moles ethylene oxide and/or from about 2 to about 50 moles propylene oxide on linear and branched fatty alcohols having from about 8 to about 15 carbon atoms in the alkyl group, the alkylphenolpolyglycolethers and/or the alcohol polypropylene glycolether and/or mixed alkylphenolpolyethers,

with a methyl or C₂-C₆ alkyl radical end group-closed addition products of from about 2 to about 50 moles of ethylene oxide and/or from about 0 to about 5 moles of propylene oxide on linear and branched fatty alcohols having from about 8 to about 30 carbon atoms on fatty acids having from about 8 to about 30 carbon atoms and on alkyl phenols having from about 8 to about 15 carbon atoms in the alkyl group, such as the types available under the trade names Dehydol® LS, Dehydo® LT (Cognis),

—C₁₂-C₃₀ fatty acid mono- and diesters of adducts of from about 1 to about 30 moles of ethylene oxide on glycerin,

adducts of from about 5 to about 60 moles of ethylene oxide on hardened castor oil,

Polyol fatty acid esters, such as the trading product Hydagen® HSP (Cognis) or Sovermol® types (Cognis),

polyalkoxylated triglycerides,

polyalkoxylated fatty acid alkyl esters of the formula (Tnio-1)

R¹CO—(OCH₂CHR²)ₙOR³ (Tnio-1)

wherein R¹CO denotes a linear branched, saturated and/or unsaturated acyl radical having from about 6 to about 22 carbon atoms, R² denotes hydrogen or methyl, R³ denotes linear or branched alkyl radicals having from about 1 to about 4 carbon atoms and w denotes numbers from about 1 to about 20,

aminoxides,

hydroxy mixed ethers, as described in DE-OS 19738866, for example,

sorbitan fatty acid esters and addition products of ethylene oxide onto sorbitan fatty acid esters such as polysorbates,

sugar fatty acid esters and addition products of ethylene oxide on sugar fatty acid esters,

adducts of ethylene oxide on fatty acid alkanolamides and fatty amines,

sugar tensides of the alkyl- and alkenyloligoglycoside type, or

sugar tensides of the fatty acid-N-alkylpolyhydroxyalkylamide type.

C₁₂-C₃₀ fatty alcohols, C₁₂-C₃₀ fatty acid triglycerides, C₁₂-C₃ₒfatty acid monoglycerides, C₁₂-C₃₀ fatty acid diglycerides and C₁₂-C₃₀fatty acid esters do not have highly-polar end groups (also evident by the low HLB values of the compounds in this group). According to the present disclosure, they are considered fatty constituents and therefore do not constitute non-ionic tensides as defined by the present disclosure.

Moreover, the agents (a) and/or (b) can additionally contain one or more non-ionic polymers.

Polymers are macromolecules having a molecular weight of at least about 1000 g/mol, preferably at least about 2500 g/mol, more preferably at least about 5000 g/mol, which includes the same, repeating organic units. Polymers are produced by polymerization of a monomer type or by polymerization of different, structurally different monomer types. If the polymer is produced by polymerization of one monomer type, it is referred to as a homopolymer. If structurally different monomer types are used in the polymerization, they are referred to as copolymers by a person skilled in the art.

The maximum molecular weight of the polymer depends on the degree of polymerization (number of polymerized monomers) and is partly determined by the polymerization method. According to the present disclosure, the maximum molecular weight of the zwitterionic polymer (d) is preferably no more than about 10⁷ g/mol, more preferably no more than about 10⁶ g/mol and even more preferably no more than about 10⁵ g/mol.

Non-ionic polymers are exemplified in that they are not charged.

Examples of suitable non-ionic polymers are vinylpyrrolidinone/vinylacrylate-copolymers, polyvinylpyrrolidinone, vinylpyrrolidinone/vinylacetate-copolymers, polyethylene glycols, ethylene/propylene/styrene-copolymers and/or butylene/ethylene/styrene-copolymers.

Further, the agents (a) and (b) according to the invention can also contain other active ingredients, excipients and additives, such as anionic, zwitterionic, amphoteric and/or cationic tensides, cationic polymers such as quaternated cellulose ether, polysiloxane with quaternary groups, dimethyldiallylammoniumchloride polymers, acrylamide-dimethyldiallyl-ammonium chloride copolymers, with diethylsulphate quaternated dimethylamino-ethylmethacrylate-vinylpyrrolidinone-copolymers, vinylpyrrolidinone-Imidazolinium-methochloride-copolymers and quaternated polyvinyl alcohol; zwitterionic and amphoteric polymers; anionic polymers, such as polyacrylic acids or cross-linked polyacrylic acids; structurants such as glucose, maleic acid and lactic acid, hair-conditioning compounds, such as phospholipids, such as lecithin and kephaline; perfume oils, dimethylisosorbide and cyclodextrine; fiber structure-improving active ingredients, more particularly mono-, di- and oligosaccharides, such as glucose, galactose, fructose and lactose; dues for coloring the agent; anti-dandruff active ingredients such as piroctone olamine, zinc omadine and climbazole; amino acids and oligopeptides; animal- and plant-based protein hydrolysates, and also in the form of their fatty acid condensation products or, if applicable, anionically or cationically modified derivatives; vegetable oils; light stabilizers and UV blockers; active ingredients such as panthenol, pantothenic acid, pantolactone, allantoin, pyrrolidinone carbonic acids and the salts thereof, as well as bisabolol; polyphenols, more particularly hydroxy cinnamic acids, 6,7-dihydroxy cumarines, hydroxy benzoic acids, catechines, tannins, leucoanthocyanidines, anthocyanidines, flavanones, flavones and flavonoles; ceramide or pseudoceramides; vitamins, provitamins and vitamin precursors; vegetable extracts; fats and waxes, such as fatty alcohols, beeswax, montan wax and paraffins; swelling agents and penetrating agents, such as glycerine, propylenglycol monoethylether, carbonates, hydrogen carbonates, guanidines, ureas, as well as primary, secondary and tertiary phosphates; opacifiers, such as latex, styrene/PVP and styrene and styrol/acrylamide copolymers; pearlescent agents, such as ethylene glycol mono and -distearate, as well as PEG-3-distearate; pigments, as well as propellants, such as propane-butane-mixtures, N₂O, dimethylether, CO₂ and air. In this context, explicit reference is made to the known monographies, e.g. Kh. Schrader, Grundlagen and Rezepturen der Kosmetika [Cosmetic principles and formulas], 2nd Edition, Hüthig Buch Verlag, Heidelberg, 1989, which reflect the corresponding knowledge of a person skilled in the art.

### Decoloration of Dyed Keratinous Fibers

The kit-of-parts as contemplated herein is a system comprising the agents (a) and (b) for the decoloration of previously dyed keratinous fibers, more particularly human hair. The dyed keratinous fibers are usually fibers which have been colored beforehand by employing conventional oxidative dyes and/or partially oxidative dyes known to a person skilled in the art.

The decolorizing agents are suitable for removing colors produced on the keratinous fibers by employing oxidizing dyes based on developer and coupler components. If the following compounds were used as developers, the colors thus produced can easily be removed effectively and almost without subsequent post-darkening by employing the decoloration agent: p-phenylendiamine, p-toluylendiamine, N,N-bis-(β-hydroxyethyl)-p-phenylendiamine, 4-N,N-bis-(β-hydroxyethyl)-amino-2-methylaniline, 2-(β-hydroxyethyl)-p-phenylendiamine, 2-(α,β-dihydroxyethyl)-p-phenylendiamine, 2-hydroxymethyl-p-phenylendiamine, bis-(2-hydroxy-5-aminophenyl)-methane, p-aminophenol, 4-amino-3-methylphenol, 2,4,5,6-tetraaminopyrimidine, 4-hydroxy-2,5,6-triaminopyrimidine, 2-hydroxy-4,5,6-triaminopyrimidine and/or 4,5-diamino-1-(β-hydroxyethyl)-pyrazole. If the following compounds were used as couplers, the colors produced thereby can likewise be removed with very good decoloration results: m-phenylendiamine derivatives, naphthols, resorcin and resorcin derivatives, pyrazolone and m-aminophenol derivatives. Suitable coupler substances are in particular 1-naphthol, 1,5-, 2,7- and 1,7-dihydroxynaphthalin, 5-amino-2-methylphenol, m-aminophenol, resorcin, resorcin monomethylether, m-phenylendiamine, 1-phenyl-3-methyl-pyrazolon-5, 2,4-dichlor-3-aminophenol, 1,3-bis-(2′,4′-diaminophenoxy)-propane, 2-chlor-resorcin, 4-chlor-resorcin, 2-chlor-6-methyl-3-aminophenol, 2-amino-3-hydroxypyridin, 2-methylresorcin, 5-methylresorcin and 2-methyl-4-chlor-5-aminophenol, 1-naphthol, 1,5-dihydroxynaphthalin, 2,7-dihydroxynaphthalin, 1,7-dihydroxynaphthalin, 3-aminophenol, 5-amino-2-methylphenol, 2-amino-3-hydroxypyridin, resorcin, 4-chlorresorcin, 2-chlor-6-methyl-3-aminophenol, 2-methylresorcin, 5-methylresorcin, 2,5-dimethylresorcin and 2,6-dihydroxy-3,4-dimethylpyridin.

The substrate to be decolored can also be dyed by employing partially-oxidizing dyes. Nitrophenylendiamines, nitroaminophenols, azo dyes, anthrachinones or indophenols are particularly suitable partially-oxidizing dyes. The preferred partially-oxidizing dyes are the compounds known under the international designations and/or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, as well as 1,4-diamino-2-nitrobenzol, 2-amino-4-nitrophenol, 1,4-bis-(β-hydroxyethyl)-amino-2-nitrobenzol, 3-nitro-4-(β-hydroxyethyl)-aminophenol, 2-(2′-hydroxyethyl)amino-4,6-dinitrophenol, 1-(2′-hydroxyethyl)amino-4-methyl-2-nitrobenzol, 1-amino-4-(2′-hydroxyethyl)-amino-5-chlor-2-nitrobenzol, 4-amino-3-nitrophenol, 1-(2′-ureidoethyl)amino-4-nitrobenzol, 4-amino-2-nitrodiphenylamine-2′-carboxylic acid, 6-nitro-1,2,3,4-tetrahydrochinoxaline, 2-Hydroxy-1,4-naphthochinon, picric acid and the salts thereof, 2-amino-6-chloro-4-nitrophenol, 4-ethylamino-3-nitrobenzoic acid and 2-chloro-6-ethylamino-1-hydroxy-4-nitrobenzol.

Moreover, the substrates to be de-colorized can also be dyed with natural dyes that occur in nature, such as Henna red, Henna neutral, Henna black, chamomile blossoms sandalwood, black tea, Cascara bark, sage, logwood, madder root, catechu, cedar and alkanna root.

The decolorizing agents as contemplated herein are designed to remove said colors and therefore themselves preferably contain no dyes, more particularly no oxidative dye precursors of the developer type and/or coupler type, as well as partially-oxidizing dyes.

In a further preferred embodiment, a kit-of-parts as contemplated herein is therefore exemplified in that
the total quantity of all dyes and oxidative precursors contained in the agent (a) is a value of maximum about 0.2 wt. %, preferably maximum about 0.1 wt. %, more preferably maximum about 0.05 wt. % and most preferably maximum about 0.01 wt. %—relative to the total weight of the agent (a)—and
the total quantity of all dyes and oxidative precursors contained in the agent (b) is a value of maximum about 0.2 wt. %, preferably maximum about 0.1 wt. %, more preferably maximum about 0.05 wt. % and most preferably maximum about 0.01 wt. %—relative to the total weight of the agent (b).

### Ready-to-Use Decolorizing Agent

The two agents (a) and (b) from the containers (A) and (B) are mixed together to produce the ready-to-use decolorizing agent, which is preferably applied to the dyed keratinous fibers and/or the hair immediately.

A second subject matter of the present disclosure is therefore a ready-to-use agent for the reductive decoloration of dyed keratinous fibers, containing
(a1) one or more reducing agents from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid) (thioglycol acid) and/or ascorbic acid, and
(b1) one or more oxidants from the group of hydrogen peroxide, potassium sulphate, sodium persulphate and/or ammonium persulphate,
wherein the weight ratio of the total quantity of all reducing agents (a1) contained in the agent to the total quantity of all oxidants (b1) contained in the agent—relative to the total weight of agent—is a value of from about 50.0 to about 4.0.

In another most preferred embodiment, the ready-to-use agent as contemplated herein contains
(a1) one or more reducing agents from the group of formamidine sulphinic acid, sodium dithionite, zinc dithionite and/or potassium dithionite, most preferably formamidine sulphinic acid.

In another most preferred embodiment, the ready-to-use agent as contemplated herein contains
(b1) hydrogen peroxide as the oxidant.

In another most preferred embodiment, the ready-to-use agent as contemplated herein is therefore exemplified in that the weight ratio from the total quantity of all reducing agents (a1) contained in the agent to the total quantity of all oxidants (b1) contained in the agent—relative to the total weight of the agent (a) plus (b)—is a value of from about 45.0 to about 5.0, preferably from about 40.0 to about 8.0, more preferably from about 30.0 to about 12.0 and most preferably from about 25.0 to about 15.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) one or more oxidants from the group of hydrogen peroxide, potassium sulphate, sodium persulphate and/or ammonium persulphate,
wherein the weight ratio of the total quantity of all reducing agents (a1) contained in the agent to the total quantity of all oxidants (b1) contained in the agent—relative to the total weight of agent—is a value of from about 50.0 to about 4.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) one or more oxidants from the group of hydrogen peroxide, potassium sulphate, sodium persulphate and/or ammonium persulphate,
wherein the weight ratio of the total quantity of all reducing agents (a1) contained in the agent to the total quantity of all oxidants (b1) contained in the agent—relative to the total weight of agent—is a value of from about 45.0 to about 5.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) one or more oxidants from the group of hydrogen peroxide, potassium sulphate, sodium persulphate and/or ammonium persulphate,
wherein the weight ratio of the total quantity of all reducing agents (a1) contained in the agent to the total quantity of all oxidants (b1) contained in the agent—relative to the total weight of agent—is a value of from about 40.0 to about 8.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) one or more oxidants from the group of hydrogen peroxide, potassium sulphate, sodium persulphate and/or ammonium persulphate,
wherein the weight ratio of the total quantity of all reducing agents (a1) contained in the agent to the total quantity of all oxidants (b1) contained in the agent—relative to the total weight of agent—is a value of from about 30.0 to about 12.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) one or more oxidants from the group of hydrogen peroxide, potassium sulphate, sodium persulphate and/or ammonium persulphate,
wherein the weight ratio of the total quantity of all reducing agents (a1) contained in the agent to the total quantity of all oxidants (b1) contained in the agent—relative to the total weight of agent—is a value of from about 25.0 to about 15.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 50.0 to about 4.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) hydrogen peroxide,
wherein the weight ratio of the total quantity of all reducing agents (a1) contained in the agent to the total quantity of all oxidants (b1) contained in the ready-to-use agent—relative to the total weight of agent—is a value of from about 45.0 to about 5.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 40.0 to about 8.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 30.0 to about 12.0.

A preferred embodiment is a ready-to-use agent for the reductive decoloration of dyed keratinous fibers, containing
(a1) one or more reducing agents from the group of formamidine sulphinic acid, sodium dithionite, zinc dithionite and/or potassium dithionite, most preferably formamidine sulphinic acid, and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 25.0 to about 15.0.

A preferred embodiment is a ready-to-use agent for the reductive decoloration of dyed keratinous fibers, containing
(a1) formamidine sulphinic acid and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 50.0 to about 4.0.

A preferred embodiment is a ready-to-use agent for the reductive decoloration of dyed keratinous fibers, containing
(a1) formamidine sulphinic acid and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 45.0 to about 5.0.

A preferred embodiment is a ready-to-use agent for the reductive decoloration of dyed keratinous fibers, containing
(a1) formamidine sulphinic acid and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 40.0 to about 8.0.

A preferred embodiment is a ready-to-use agent for the reductive decoloration of dyed keratinous fibers, containing
(a1) formamidine sulphinic acid and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 30.0 to about 12.0.

A preferred embodiment is a ready-to-use agent for the reductive decoloration of dyed keratinous fibers, containing
(a1) formamidine sulphinic acid and
(b1) hydrogen peroxide,
wherein the weight ratio of the total quantity of all reducing agents (a1) contained in the agent to the total quantity of all oxidants (b1) contained in the agent—relative to the total weight of agent—is a value of from about 25.0 to about 15.0.

With respect to further preferred embodiments of the ready-to-use decolorizing agents, of the reducing agents (a1), of the oxidants (b1) and of the weight ratio of the total quantity of all reducing agents (a1) contained in the agent to the total quantity of all oxidants (b1) contained in the agent, the statements made about the kit-of-parts apply mutatis mutandis.

All further most preferred embodiments are those already described for the kit-of-parts as contemplated herein.

### Method

The kit-of-parts as contemplated herein described above can be used for reductive decoloration.

A third subject of the present disclosure is a method for the reductive decoloration of dyed keratinous fibers, including the following steps in the specified sequence
(I) Production of a ready-to-use decolorizing agent by mixing a first agent (a) with a second agent (b), wherein
   the agent (a) is an agent, as disclosed in detail in the description of the first subject matter as contemplated herein, and
   the agent (b) is an agent, as disclosed in detail in the description of the first subject matter as contemplated herein,
(II) Application of the ready-to-use decolorizing agent on dyed keratinous fibers,
(III) Leaving the decolorizing agent to take effect
(IV) Rinsing the decolorizing agent out of the keratinous fibers.

In other words, a third subject of the present disclosure is a method for the reductive decoloration of dyed keratinous fibers, including the following steps in the specified sequence
(I) Production of a ready-to-use decolorizing agent by mixing a first agent (a) with a second agent (b), wherein
   the agent (a)
(a1) contains one or more reducing agents from the group of formamidine sulphinic acid, sodium dithionite, zinc dithionite, potassium dithionite, hydroxymethane sulphinic acid, aminomethane sulphinic acid, cysteine, thio lactic acid, sulfanyl acetic acid (thioglycol acid) and/or ascorbic acid, and
   the agent (b)
(b1) contains one or more oxidants from the group of hydrogen peroxide, potassium sulphate, sodium persulphate and/or ammonium persulphate, and
   in the ready-to-use agent, the weight ratio of the total quantity of all reducing agents (a1) to the total quantity of all oxidants (b1)—relative to the total weight of the ready-to-use agent—is a value of from about 50.0 to about 4.0,
(II) Application of the ready-to-use decolorizing agent on dyed keratinous fibers,
(III) Leaving the decolorizing agent to take effect
(IV) Rinsing the decolorizing agent out of the keratinous fibers.

In Step (I), the ready-to-use decolorizing agent is produced by mixing the agents (a) and (b). The agents (a) and (b) are the two agents of the kit-of-parts as contemplated herein. With respect to other preferred embodiments of the agents (a) and (b), the statements made on the kit-of-parts as contemplated herein apply mutatis mutandis.

In Step (II), the ready-to-use agent produced by mixing agents (a) and (b) is applied to the keratinous fibers and/or to the hair. With respect to further preferred embodiments of the ready-to-use agent, the statements made about the kit-of-parts as contemplated herein apply mutatis mutandis.

The exothermic reaction described above, which is initiated by mixing the agents (a) and (b) causes the ready-to-use agent to heat up. This heating ensures the complete dissolution of the reducing agent (a1), and can also be used to further intensify the decoloration result. The tests carried out as part of the present disclosure have shown that applying the heated agent (compared to applying the agent at room temperature) leads to a further improvement in the decoloration result. To exploit this further advantage, it is most preferable for a period from about 10 seconds to about 30 minutes, preferably from about 10 seconds to about 15 minutes and most preferably from about 10 seconds to about 10 minutes to lapse between steps (I) and (II). In other words, it is preferable for a maximum of about 30 minutes, more preferable about 15 minutes and most preferable about 10 minutes to lapse between producing the ready-to-use agent and applying it to the hair.

Most preferred in a further embodiment is a method, which is exemplified in that a period from about 10 seconds to about 30 minutes, preferably from about 10 seconds to about 15 minutes and most preferably from about 10 seconds to about 10 minutes lapses between steps (I) and (II).

Once the ready-to-use decolorizing agent has been applied to the keratinous fibers (Step (II), the ready-to-use decolorizing agent is left to take effect on the keratinous fibers. The application period can be selected based on the desired decoloration effect. For example, the decolorizing agent can be left to take effect on the hair for a period of from about 5 to about 60 minutes, preferably from about 10 to about 55 minutes, more preferably from about 20 to about 50 minutes, most preferably from about 30 to about 45 minutes,

Most preferred in a further embodiment is a method, exemplified in that the agents (a) and (b) are mixed with one another in a quantity ratio (a)/(b) from about 0.1 to about 1.0, preferably from about 0.15 to about 0.9, more preferably from about 0.18 to about 0.8 and most preferably from about 0.2 to about 0.5.

Most preferred in a further embodiment is a method, exemplified in that the mixture of agent (a) and (b) (i.e. the ready-to-use decolorizing agent) is hydrous and has a pH value from about 7.5 to about 12.5, preferably from about 8.0 to about 11.5, more preferably from about 8.5 to about 10.5 and most preferably from about 8.5 to about 9.5.

With respect to other preferred embodiments of the method as contemplated herein, the statements made regarding the kit-of-parts and the ready-to use agent as contemplated herein apply mutatis mutandis.

### EXAMPLES

### 1.1. Coloration

The following formulations were produced (all data in wt. %):

### Dye Cream (F1)

| Raw material | wt. % |
|---|---|
| Cetearyl alcohol | 8.5 |
| C12-C18 fatty alcohols | 3.0 |
| Ceteareth-20 | 0.5 |
| Ceteareth-12 | 0.5 |
| Plantacare 1200 UP (laurylglucoside, 50-53% hydrous solution) | 2.0 |
| Sodium laureth-6 carboxylate (21% i hydrous solution) | 10.0 |
| Sodium myreth sulfate (68-73% hydrous solution) | 2.8 |
| Sodium acrylate, trimethylammoniopropylacrylamide chloride | 3.8 |
| copolymer | |
| (19-21% hydrous solution) | |
| Potassium hydroxide | 0.83 |
| p-toluylendiamine, sulfate | 2.25 |
| m-aminophenol | 0.075 |
| 2-amino-3-methylphenol | 0.12 |
| Resorcin | 0.62 |
| 4-chlorresorcin | 0.26 |
| 3-amino-2-methylamino-6-methoxypyridin | 0.04 |
| 1,3-bis(2,4-diaminophenoxy)propane, tetrahydrochloride | 0.05 |
| Ammonium sulfate | 0.1 |
| Sodium sulfate | 0.4 |
| Ascorbic acid | 0.1 |
| 1-Hydroxyethane-1,1-diphosphonic acid (60% hydrous solution) | 0.2 |
| Ammonia (25% hydrous solution) | 7.2 |
| Water | Ad 100 |

### Oxidant (Ox)

| Raw material | wt. % |
|---|---|
| Sodium benzoate | 0.04 |
| Dipicolinic acid | 0.1 |
| Di-sodium pyrophosphate | 0.1 |
| Potassium hydroxide | 0.09 |
| 1,2-Propylenglycol | 1.0 |
| 1-Hydroxyethane-1,1-diphosphonic acid (60% hydrous solution) | 0.25 |
| Paraffinum liquidum | 0.30 |
| Steartrimonium chloride | 0.39 |
| Cetearyl alcohol | 3.4 |
| Ceteareth-20 | 1.0 |
| Hydrogen peroxide (50% hydrous solution) | 12.0 |

The dye cream (F1) and the oxidant (Ox) were then mixed in a ratio of 1:1 and applied to the hair strands (Kerling Euro natural hair, white). The weight ratio of application mixture to hair was 4:1, exposure time 30 minutes at a temperature of 32 degrees Celsius. The strands were then rinsed with water, dried and left to rest at room temperature for at least 24 hours. The strands were then dyed in a dark brown shade.

### 1.2 Decolorization

The containers (A) and (B) were filled with the following agents (all values in grams):

### Container (A) Containing 20 g of Agent (a)

| | Agent (a) |
|---|---|
| Versagel M 1600⁽¹⁾ | 3.58 g |
| Sodium metasilicate (anhydrous, fine) | 2.20 g |
| Formamidine sulphinic acid | 8.00 g |
| Paraffinum Liquidum | 20.00 g |

⁽¹⁾Versagel M 2600: INCI Paraffinum Liquidum (Mineral Oil), Ethylene/Propylene/Styrene-Copolymer, Butylene/Ethylene/Styrene-Copolymer

### Container (B) Containing 80 g Agent (b)

| | Agent (bV) | Agent (bE) |
|---|---|---|
| Agent (b) | Comparison | Present Disclosure |
| Cetearyl alcohol | 4.0 g | 4.0 g |
| PEG-40 Hydrogenated Castor Oil | 0.80 g | 0.80 g |
| Sodium laureth sulphate (C₁₂-C₁₄, 2 | 0.54 g | 0.54 g |
| EO) | | |
| Hydrogen peroxide | — | 0.40 g |
| Dipicolinic acid | 0.032 g | 0.032 g |
| Hydroxy ethane 1.1 diphosphonic | 0.12 g | 0.12 g |
| acid | | |
| Water | 80 g | 80 g |

The ready-to-use decolorizing agent was produced by mixing 20 g of the agent (a) with 80 g of the agent (b) by stirring at room temperature.

For each application mixture, the temperature was measured and the time after which the reducing agent (formamidine sulphinic acid) had completely dissolved was determined:

| | Comparison | Present Disclosure |
|---|---|---|
| | 20 g agent (a) + | 20 g agent (a) + 80 g agent |
| | 80 agent (bV) | (bE) |
| Temperature stirring for | 20° C. | 31° C. |
| 20 seconds | | |
| Period until complete | 240 seconds | 60 seconds |
| dissolution of the | | |
| formamidine sulphinic | | |
| acid | | |

As soon as the reducing agent (formamidine sulphinic acid) in the ready-to-use decolorizing agent (agent (a) plus (b)) had completely dissolved, the decolorizing agent as contemplated herein is applied to the previously dyed hair strands, left at room temperature for 60 minutes and then rinsed out again. The hair strands were then dried.
The coloration of the strands thus decolored was assessed visually. The color intensity was assessed on the following scale:
0—strands have no longer have any perceptible color (white-blond, as the original color of the used Kerling Euro natural hair, white)
1—Strands with weak color intensity
1—Strands with medium color intensity
3—Strands with strong color intensity
4—Color of the stands as immediately after dyeing, no decoloration effect

| | Comparison | Present Disclosure |
|---|---|---|
| | 20 g agent (a) + 80 | 20 g agent (a) + 80 g |
| | agent (bV) | agent (bE) |
| Coloration of strands after | 2 | 1 |
| applying and washing out | | |
| the decolorizing agent | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Kit-of-parts for the reductive decoloration of dyed keratinous fibers comprising, packaged separately from one another
(I) a first container (A) comprising a cosmetic agent (a) and
(II) a second container (B) comprising a cosmetic agent (b),
wherein
the agent (a) in the first container (A) comprises
(a1) one or more reducing agents chosen from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid, and/or ascorbic acid, and
the agent (b) in container (B) comprises
(b1) one or more oxidants chosen from the group of hydrogen peroxide, potassium sulphate, sodium persulphate and/or ammonium persulphate, and
wherein the weight ratio of the total quantity of all reducing agents (a1) contained in agent (a) to the total quantity of all oxidants (b1) contained in agent (b)—relative to the total weight of agents (a) plus (b)—is a value of from about 50.0 to about 4.0.

2. Kit-of-parts according to claim 1, wherein the agent (a) in the first container (A) comprises
(a1) one or more reducing agents chosen from the group of formamidine sulphinic acid, sodium dithionite, zinc dithionite and/or potassium dithionite.

3. Kit-of-parts according to claim 1, wherein the agent (a) in the first container (A) comprises—relative to the total weight of the agent (a)—one or more reducing agents (a1) in a total quantity of from about 5.0 to about 80.0 wt. %.

4. Kit-of-parts according to claim 1, wherein the agent (a) in the first container (A) has—relative to the total weight of the agent (a)—a water content of below about 10.0 wt. %.

5. Kit-of-parts according to claim 1, wherein the agent (a) in the first container (A) further comprises—relative to the total weight of the agent (a)—one or more fatty constituents (a2) chosen from the group of C₁₂-C₃₀ fatty alcohols, C₁₂-C₃₀ fatty acid triglycerides, C₁₂-C₃ₒfatty acid monoglycerides, C₁₂-C₃₀ fatty acid diglycerides, C₁₂-C₃₀ fatty acid esters, hydrocarbons and/or silicone oils in a total quantity of from about 10 to about 90 wt. %.

6. Kit-of-parts according to claim 1, wherein the agent (a) in the first container (A) further comprises
(a3) one or more alkalizing agents chosen from the group of sodium metasilicate, potassium metasilicate, potassium hydroxide, sodium hydroxide, sodium carbonate, potassium carbonate, sodium hydrogen carbonate and/or potassium hydrogen carbonate.

7. Kit-of-parts according to claim 1, wherein the agent (a) comprises—relative to the total weight of the agent (a)—from about 3.0 to about 25.0 wt. %, sodium metasilicate.

8. Kit-of-parts according to claim 1, wherein the agent (b) in the second container (B) comprises
(b1) contains hydrogen peroxide as the oxidant.

9. Kit-of-parts according to claim 1, wherein the agent (b) in the first container (B) comprises—relative to the total weight of the agent (b)—one or more oxidants (b1) in a total quantity of from about 1.0 to about 5.0 wt. %.

10. Kit-of-parts according to claim 1, wherein the agent (b) comprises—relative to the total weight of the agent (b)—from about 30 to about 97 wt. % of water.

11. Kit-of-parts according to claim 1, wherein the weight ratio from the total quantity of all reducing agents (a1) contained in the agent (a) to the total quantity of all oxidants (b1) contained in the agent (b)—relative to the total weight of the agent (a) plus (b)—is a value of from about 45.0 to about 5.0.

12. Kit-of-parts according to claim 1, wherein the containers (A) and (B) comprise the agents (a) and (b) in such quantities that the quantity ratio of agent (a) to agent (b) is a value of from about 0.1 to about 1.0.

13. Kit-of-parts according to claim 1, consisting of two agents (a) and (b) in the two respective containers (A) and (B).

14. Ready-to-use agent for the reductive decoloration of dyed keratinous fibers, comprising
(a1) one or more reducing agents chosen from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid, and/or ascorbic acid, and
(b1) one or more oxidants chosen from the group of hydrogen peroxide, potassium sulphate, sodium persulphate and/or ammonium persulphate,
wherein the weight ratio of the total quantity of all reducing agents (a1) contained in the agent to the total quantity of all oxidants (b1) contained in the agent—relative to the total weight of agent—is a value of from about 50.0 to about 4.0.

15. Agent according to claim 14, comprising
(a1) one or more reducing agents from the group of formamidine sulphinic acid, sodium dithionite, zinc dithionite and/or potassium dithionite, most preferably formamidine sulphinic acid.

16. Agent according to claim 14, comprising
(b1) hydrogen peroxide as the oxidant.

17. Agent according to claim 14, wherein the weight ratio from the total quantity of all reducing agents (a1) contained in the agent to the total quantity of all oxidants (b1) contained in the agent—relative to the total weight of the agent (a) plus (b)—is a value of from about 45.0 to about 5.0.

18. Method for the reductive decoloration of dyed keratinous fibers, comprising the following steps in the stated sequence
(I) producing a ready-to-use decolorizing agent by mixing a first agent (a) with a second agent (b), wherein
agent (a) comprises
(a1) one or more reducing agents chosen from the group of formamidine sulphinic acids, sodium dithionite, zinc dithionite, potassium dithionite, hydroxymethane sulphinic acid, aminomethane sulphinic acid, cysteine, thiolactic acid, sulfanyl acetic acid, and/or ascorbic acid, and
agent (b) comprises
(b1) one or more oxidants chosen from the group of hydrogen peroxide, potassium sulphate, sodium persulphate and/or ammonium persulphate,
(II) applying the ready-to-use decolorizing agent on dyed keratinous fibers,
(III) allowing the decolorizing agent to take effect,
(IV) rinsing the decolorizing agent out of the keratinous fibers.

19. Method according to claim 18, wherein a period from 10 seconds to 30 minutes lapses between steps (I) and (II).

20. Method according to claim 18, wherein the agents (a) and (b) are mixed with one another in a quantity ratio (a)/(b) from about 0.1 to about 1.0, wherein the mixture of agent (a) and (b) is hydrous, and wherein the mixture of agent (a) and (b) has a pH value from about 7.5 to about 12.5.

21. (canceled)
